(19) United States
(12) Patent Application Publication
(10) Pub. No.: US 2025/0171590 A1
(43) Pub. Date: May 29, 2025

(54) POLYIMIDE PRECURSOR FOR DISPLAY SUBSTRATE, POLYIMIDE FILM FOR DISPLAY SUBSTRATE, AND DISPLAY SUBSTRATE

(71) Applicant: UBE Corporation, Ube-shi (JP)

(72) Inventors: Taiga ADACHI, Ube-shi (JP); Norio MIURA, Ube-shi (JP); Sousuke HONMA, Ube-shi (JP); Nobu IIZUMI, Ube-shi (JP); Shohei INOUE, Ube-shi (JP); Nobuharu HISANO, Ube-shi (JP)

(21) Appl. No.: 18/839,695

(22) PCT Filed: Mar. 3, 2023
(86) PCT No.: PCT/JP2023/008169

§ 371 Date: Aug. 19, 2024

(30) Foreign Application Priority Data
Mar. 3, 2022 (JP) 2022-032805
Mar. 3, 2022 (JP) 2022-032822
Mar. 3, 2022 (JP) 2022-032838

(51) Int. Cl. C08G 73/06; C08G 73/10; C08G 73/16; C08J 5/18

(52) U.S. Cl. CPC C08G 73/0644; C08G 73/1078; C08G 73/16; C08J 5/18; C08J 2379/08

(57) ABSTRACT

A polyimide precursor for a display substrate capable of forming a polyimide film for a display substrate having a charge half-life of 48 seconds or more and/or a charge decay ratio after 120 seconds of 63% or less in measurement of the charge half-life in accordance with JIS L 1094A.

### TECHNICAL FIELD

The present invention relates to a polyimide precursor for a display substrate which can contribute to a suppression in charge up and can achieve high adhesion, a polyimide film for a display substrate formed from this, and a display substrate.

### BACKGROUND ART

Polyimides, which have excellent heat resistance, solvent resistance (chemical resistance), mechanical properties, electrical properties, and the like, are widely used in applications to electrical and electronic devices, such as flexible wiring substrates and tape automated bonding (TAB) tapes. For example, polyimides prepared from an aromatic tetracarboxylic dianhydride and an aromatic diamine, particularly, a polyimide prepared from 3,3′,4,4′-biphenyltetracarboxylic dianhydride and paraphenylenediamine is suitably used.

Moreover, polyimides have been examined as an alternative to glass substrates in the display device field. By replacing glass substrates by plastic substrates made of polyimide in display substrates used in a variety of display devices, displays which have light weight and high flexibility and can be bent or rolled can be provided.

For example, Patent Document 1 proposes a method using a polyimide precursor containing a specific unit structure as a polyimide precursor applicable to display substrate applications.

### RELATED ART DOCUMENTS

### Patent Documents

Patent Document 1: JP 2012-41531 A

### SUMMARY OF THE INVENTION

### Problem to be Solved by the Invention

In polyimide films used in display substrate applications, to obtain sufficient gas barrier properties, usually, these polyimide films are used in the state where an inorganic gas barrier layer made of SiOₓ or the like is formed on their surfaces. On the other hand, use of polyimide films in display substrate applications has a problem of charge up caused by charges accumulated at an interface between the polyimide film and the inorganic gas barrier layer, in which charge up causes a slight amount of current to flow in a switching element or the like, thereby causing an afterglow on a display.

### Means for Solving Problems

The present inventors, who have conducted extensive research about this problem, have found that in use of polyimide films in display substrate applications, by using a polyimide film for a display substrate having an increased charge half-life or a reduced charge decay ratio after 120 seconds, cancellation of charge accumulation at the interface between a polyimide film and an inorganic gas barrier layer is promoted, and thus charge up is suppressed, and have completed the present invention.

The present inventors, who have conducted extensive research, have also found that high adhesion can be achieved by use of such a configuration.

Specifically, the present invention provides [1] to [9] below.

[1] A polyimide precursor for a display substrate, the polyimide precursor being capable of forming a polyimide film for a display substrate having a charge half-life of 48 seconds or more in measurement of the charge half-life in accordance with A JIS L 1094A.

[2] A polyimide precursor for a display substrate, the polyimide precursor being capable of forming a polyimide film for a display substrate having a charge decay ratio after 120 seconds of 63% or less in measurement of the charge half-life in accordance with JIS L 1094A.

[3] The polyimide precursor for a display substrate according to [1], which is a polyimide precursor for a display substrate, the polyimide precursor being capable of forming a polyimide film for a display substrate having a charge decay ratio after 120 seconds of 63% or less in the measurement of the charge half-life in accordance with JIS L 1094A.

[4] The polyimide precursor for a display substrate according to any one of [1] to [3], wherein the polyimide precursor has a structural unit represented by General Formula (2) below, and contains a group represented by General Formula (3) below as Y² in General Formula (2) below:

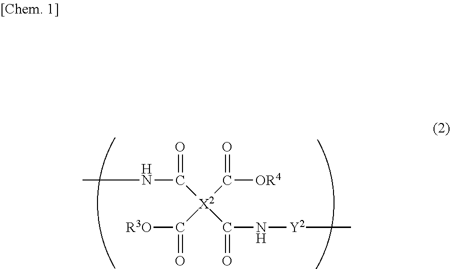

where in General Formula (2) above, X² is a tetravalent aromatic or aliphatic group, Y² is a divalent aromatic group, and R³ and R⁴ are each independently a hydrogen atom, a C₁ to C₆ alkyl group, or a C₃ to C₉ alkylsilyl group;

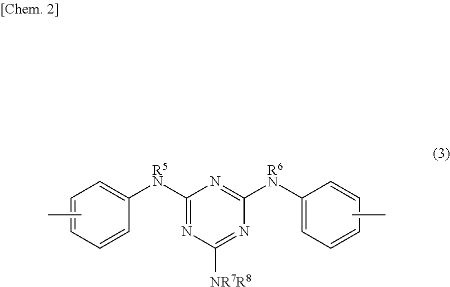

where in General Formula (3) above, R⁵, R⁶, R⁷, and R⁸ are each independently a hydrogen atom, an optionally substituted C₁ to C₁₂ alkyl group, or an optionally substituted C₆ to C₁₂ aryl group.

[5] The polyimide precursor for a display substrate according to [4], wherein the polyimide precursor contains a group represented by General Formula (5) below as the group represented by General Formula (3):

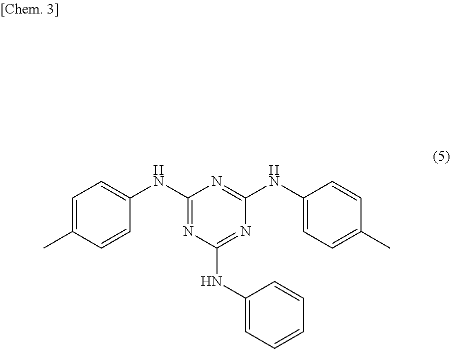

[6] The polyimide precursor for a display substrate according to [4] or [5], wherein the proportion of the structural unit containing the group represented by General Formula (3) as the group represented by Y² in the structural unit represented by General Formula (2) constituting the polyimide precursor for a display substrate is 50 to 100 mol %.

[7] A polyimide precursor composition comprising the polyimide precursor for a display substrate according to [1] and at least one inorganic oxide selected from the group consisting of SiO₂, Al₂O₃, TiO₂, PbTiO₃, CaTiO₃, SrTiO₃, CaZrO₃, and BaTiO₃,
wherein the polyimide precursor for a display substrate is a polyimide precursor having a structural unit represented by General Formula (6) below:

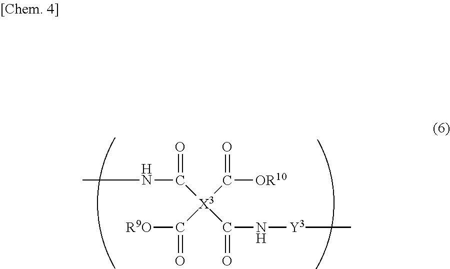

where in General Formula (6) above, X³ is a tetravalent aromatic or aliphatic group, Y³ is a divalent aromatic group, and R⁹ and R¹⁰ are each independently a hydrogen atom, a C₁ to C₆ alkyl group, or a C₃ to C₉ alkylsilyl group.

[8] A polyimide film for a display substrate, which is formed from the polyimide precursor for a display substrate according to any one of [1] to [6] or the polyimide precursor composition for a display substrate according to [7].

[9] A display substrate comprising the polyimide film for a display substrate according to [8].

### Effects of Invention

The present invention provides a polyimide precursor for a display substrate which can contribute to a suppression in charge up and can achieve high adhesion.

### DESCRIPTION OF EMBODIMENTS

### <Polyimide Precursor for Display Substrate>

The polyimide precursor for a display substrate according to the present invention is a polyimide precursor capable of forming a polyimide for a display substrate, and more specifically is a polyimide precursor capable of forming a polyimide film for a display substrate having a charge half-life of 48 seconds or more in measurement of the charge half-life in accordance with JIS L 1094A.

For the polyimide precursor for a display substrate according to the present invention, it is sufficient that the polyimide film formed from the polyimide precursor has a charge half-life of 48 seconds or more in the measurement of the charge half-life in accordance with JIS L 1094A. To more appropriately suppress charge up, the charge half-life is preferably 50 seconds or more, more preferably 52 seconds or more. The charge half-life in the measurement of the charge half-life refers to the time until the charge amount halves after the polyimide film is charged by corona discharge.

The charge half-life can be measured as follows, for example. Specifically, the polyimide precursor for a display substrate according to the present invention is subjected to an imidization reaction by a known method such as chemical or thermal imidization to form a polyimide film. The obtained polyimide film is charged by corona discharge in accordance with JIS L 1094A, and then the charge half-life is measured. Thus, the charge half-life can be measured. For example, in the case where the imidization reaction is performed by thermal imidization, a method can be used, in which thermal imidization is performed by heating the polyimide precursor under a nitrogen atmosphere to a temperature of preferably 350° C. or higher and 500° C. or lower, typically to 450° C., and the charge half-life of the obtained polyimide film is measured in accordance with JIS L 1094A.

Alternatively, the polyimide precursor for a display substrate according to another embodiment of the present invention is a polyimide precursor capable of forming a polyimide for a display substrate, in which a polyimide film formed from the polyimide precursor has a charge decay ratio after 120 seconds of 63% or less in the measurement of the charge half-life in accordance with JIS L 1094A.

For the polyimide precursor for a display substrate according to another embodiment of the present invention, it is sufficient that the polyimide film formed from the polyimide precursor has a charge decay ratio after 120 seconds of 63% or less in the measurement of the charge half-life in accordance with JIS L 1094A. To more appropriately suppress charge up, the charge decay ratio after 120 seconds is preferably 61% or less. The lower limit of the charge decay ratio after 120 seconds is preferably 30% or more. The charge decay ratio after 120 seconds in the measurement of the charge half-life is a proportion of an amount of the charge amount reduced after 120 seconds from charging of the polyimide film by corona discharge to the charge amount immediately after the charging. In other words, the charge decay ratio after 120 seconds in the measurement of the charge half-life is calculated from the equation: charge decay ratio after 120 seconds (%)={(charge amount immediately after charging−charge amount after 120 seconds)+charge amount immediately after charging}×100.

The charge decay ratio after 120 seconds can be measured as follows, for example. Specifically, the polyimide precursor for a display substrate according to the present invention is subjected to an imidization reaction by a known method such as chemical or thermal imidization to form a polyimide film. The obtained polyimide film is charged by corona discharge in accordance with JIS L 1094A, and then the charge amount immediately after the charging and the charge amount after 120 seconds are measured. At this time, these charge amounts may be measured simultaneously with the measurement of the charge half-life. For example, in the case where the imidization reaction is performed by thermal imidization, a method can be used, in which thermal imidization is performed by heating the polyimide precursor under a nitrogen atmosphere to a temperature of preferably 350° C. or higher and 500° C. or lower, typically to 450° C., and the charge decay ratio after 120 seconds of the obtained polyimide film is measured in accordance with JIS L 1094A.

In the polyimide precursor for a display substrate according to the present invention, preferably, the polyimide film formed from the polyimide precursor has both a charge half-life in the measurement of the charge half-life in accordance with JIS L 1094A and a charge decay ratio after 120 seconds in the measurement of the charge half-life in accordance with JIS L 1094A within the above ranges. Such a configuration can more appropriately suppress charge up.

In the present invention, from the viewpoint of more appropriately suppressing charge up, the polyimide firm, when used in display substrate applications, preferably demonstrates the charge half-life or charge decay ratio after 120 seconds close to that of an inorganic material which forms an inorganic layer as a gas barrier layer laminated on the polyimide film for a display substrate. In particular, SiOₓ is suitably used as the inorganic material which forms the inorganic layer. Since the inorganic material such as SiOₓ has a relatively long charge half-life and a relatively small charge decay ratio after 120 seconds, the charge half-life or charge decay ratio after 120 seconds of the polyimide film can be made closer to those of the inorganic material such as SiOₓ by further increasing the charge half-life and further reducing the charge decay ratio after 120 seconds as described above. Thereby, the charge up can be more appropriately suppressed. The charge half-life and the charge decay ratio after 120 seconds of the inorganic material which forms the inorganic layer can be determined by the same methods as those in the measurement of the charge half-life in accordance with JIS L 1094A and the measurement of the charge decay ratio after 120 seconds described above.

In the polyimide precursor for a display substrate according to the present invention, the polyimide film formed from the polyimide precursor has a 90° peel strength of preferably 15 mN/mm or more, more preferably 20 mN/mm or more. The 90° peel strength can be measured by performing an imidization reaction on the polyimide precursor for a display substrate according to the present invention by a known method such as chemical or thermal imidization to form a polyimide film on the surface of glass, and performing a 90° peeling test on the formed polyimide film. For example, in the case where the imidization reaction is performed by thermal imidization, thermal imidization can be performed under a nitrogen atmosphere by heating to a temperature of preferably 350° C. or higher and 500° C. or lower, typically to 450° C.

For the polyimide precursor for a display substrate according to the present invention, it is sufficient that the polyimide film formed from the polyimide precursor has the charge half-life or the charge decay ratio after 120 seconds within the above ranges, and is not particularly limited. Suitable examples thereof include polyimide precursors for a display substrate according to a first embodiment, a second embodiment, and a third embodiment described below.

### <Polyimide Precursor for Display Substrate According to First Embodiment>

The polyimide precursor for a display substrate according to a first embodiment (hereinafter, referred to as “polyimide precursor according to the first embodiment”) is a polyimide precursor having a structural unit represented by General Formula (1) below, wherein a group containing an acidic group is contained as at least part of a group represented by X¹ in General Formula (1) below, a group represented by Y¹ in General Formula (1) below, and a terminal group, and the acidic group content ratio is 15× 10⁻³ or more:

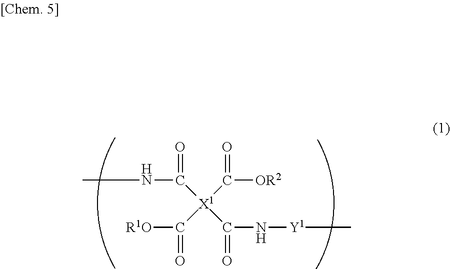

where in General Formula (1), X¹ is a tetravalent aromatic or aliphatic group, Y¹ is a divalent aromatic group, R¹ and R² are each independently a hydrogen atom, a C₁ to C₆ alkyl group, or a C₃ to C₉ alkylsilyl group.

In General Formula (1), X¹ is a residue of a tetracarboxylic acid from which four COOH groups are removed (i.e., a residue of a tetracarboxylic dianhydride from which two carboxylic anhydride groups (CO)₂O are removed), and Y¹ is a residue of a diamine from which two NH₂ groups are removed. R¹ and R² are preferably a hydrogen atom or a C₃ to C₉ alkylsilyl group, and are more preferably a hydrogen atom.

Examples of the structural unit represented by General Formula (1) above include those obtained by reacting a tetracarboxylic acid component with a diamine component to form amido bonds (—CONH—).

Examples of the tetracarboxylic acid component include aromatic tetracarboxylic dianhydrides, aliphatic tetracarboxylic dianhydrides, and the like.

Specific examples of aromatic tetracarboxylic dianhydrides include 3,3′,4,4′-biphenyltetracarboxylic dianhydride (s-BPDA), pyromellitic dianhydride, 2,3,3′,4′-biphenyltetracarboxylic dianhydride, 4,4′-oxydiphthalic dianhydride, diphenylsulfone-3,4,3′,4′-tetracarboxylic dianhydride, bis(3,4-dicarboxyphenyl) sulfide dianhydride, 2,2-bis(3,4-dicarboxyphenyl)-1,1,1,3,3,3-hexafluoropropane dianhydride (another name: 4,4′-(hexafluoroisopropylidene)diphthalic anhydride), 2,3,3′,4′-benzophenonetetracarboxylic dianhydride, 3,3′,4,4′-benzophenonetetracarboxylic dianhydride, bis(3,4-dicarboxyphenyl) methane dianhydride, 2,2-bis(3,4-dicarboxyphenyl) propane dianhydride, p-phenylenebis(trimellitic acid monoester acid anhydride), p-biphenylenebis(trimellitic acid monoester acid anhydride), m-terphenyl-3,4,3′,4′-tetracarboxylic dianhydride, p-terphenyl-3,4,3′,4′-tetracarboxylic dianhydride, 1,3-bis(3,4-dicarboxyphenoxy)benzene dianhydride, 1,4-bis(3,4-dicarboxyphenoxy)benzene dianhydride, 1,4-bis(3,4-dicarboxyphenoxy) biphenyl dianhydride, 2,2-bis[(3,4-dicarboxyphenoxy)phenyl]propane dianhydride, 2,3,6,7-naphthalenetetracarboxylic dianhydride, 1,4,5,8-naphthalenetetracarboxylic dianhydride, 4,4′-(2,2-hexafluoroisopropylidene)diphthalic dianhydride, and the like. These may be used alone or as a mixture thereof.

The aliphatic tetracarboxylic dianhydrides suitably used are alicyclic tetracarboxylic dianhydrides. Specific examples of such alicyclic tetracarboxylic dianhydrides include (1S,2R,4S,5R)-cyclohexanetetracarboxylic dianhydride, cis,cis,cis-1,2,4,5-cyclohexanetetracarboxylic dianhydride, (1S,2S,4R,5R)-cyclohexanetetracarboxylic dianhydride, (1R,2S,4S,5R)-cyclohexanetetracarboxylic dianhydride, bicyclo[2.2.2]octane-2, 3, 5,6-tetracarboxylic dianhydride, bicyclo[2.2.2]oct-7-ene-2,3,5,6-tetracarboxylic dianhydride, 4-(2,5-dioxotetrahydrofuran-3-yl)-tetralin-1,2-dicarboxylic anhydride, tetrahydrofuran-2, 3, 4, 5-tetracarboxylic dianhydride, bicyclo-3,3′,4,4′-tetracarboxylic dianhydride, 1,2,3,4-cyclopentanetetracarboxylic dianhydride, 1,2,3,4-cyclobutanetetracarboxylic dianhydride (hereinafter, also referred to as “CBDA” in some cases), 1,3-dimethyl-1,2,3,4-cyclobutanetetracarboxylic dianhydride, 1,4-dimethyl-1,2,3,4-cyclobutanetetracarboxylic dianhydride, 1,2,3,4-cyclohexanetetracarboxylic dianhydride, pentacyclo[8.2.1.1^{4,7}.0^{2,9}.0^{3,8}]tetradecane-5,6,11,12-tetracarboxylic dianhydride, 5-(2,5-dioxotetrahydrofuryl)-3-methyl-3-cyclohexene-1,2-dicarboxylic anhydride, cyclohex-1-ene-2,3,5,6-tetracarboxylic dianhydride, bicyclo[2.2.1]heptane-2,3,5,6-tetracarboxylic dianhydride, and the like. These may be used alone or as a mixture thereof.

As tetracarboxylic acid compounds as the tetracarboxylic acid component, acid dianhydrides selected from the group consisting of 3,3′,4,4′-biphenyltetracarboxylic dianhydride (s-BPDA), pyromellitic dianhydride (PMDA), 4,4′-oxydiphthalic dianhydride (ODPA), 3,3′,4,4′-benzophenonetetracarboxylic dianhydride (BTDA), 1,2,3,4-cyclobutanetetracarboxylic dianhydride (CBDA), 2,2-bis(3,4-dicarboxyphenyl)-1,1,1,3,3,3-hexafluoropropane dianhydride (6-FDA), and 3,3′,4,4′-diphenylsulfonetetracarboxylic dianhydride (DSDA) are preferred, and 3,3′,4,4′-biphenyltetracarboxylic dianhydride (s-BPDA) is more preferred.

Examples of diamine compounds as the diamine component include aromatic diamines having an aromatic group, such as 4,4′-diaminodiphenyl ether, 2,2′-dimethylbenzidine, 4,4′-diaminodiphenylmethane, 4,4′-diamino-1,2-diphenylethane, p-phenylenediamine (PPD), m-phenylenediamine, 2,4-diaminotoluene, 1,3-bis(4-aminophenoxy)benzene, 1,4-bis(4-aminophenoxy)benzene, 2,2-bis[4-(4-aminophenoxy)phenyl]propane, m-xylylenediamine, p-xylylenediamine, 2,2-bis[4-(4-aminophenoxy phenyl]propane, 4,4′-methylenebis(2,6-xylidine), α,α′-bis(4-aminophenyl)-1,4-diisopropylbenzene, 2,2′-dimethyl-4,4′-aminobiphenyl, 3,3′-dimethyl-4,4′-aminobiphenyl, and 2,2′-ethylenedianiline; alicyclic diamines having an alicyclic structure, such as 1,4-diaminocyclohexane, 1,4-diamino-2-methylcyclohexane, 1,4-diamino-2-ethylcyclohexane, 1,4-diamino-2-n-propylcyclohexane, 1,4-diamino-2-isopropylcyclohexane, 1,4-diamino-2-n-butylcyclohexane, 1,4-diamino-2-isobutylcyclohexane, 1,4-diamino-2-sec-butylcyclohexane, 1,4-diamino-2-tert-butylcyclohexane, 1,2-diaminocyclohexane, 1,3-diaminocyclobutane, 1,4-bis(aminomethyl)cyclohexane, 1,3-bis(aminomethyl)cyclohexane, diaminobicycloheptane, diaminomethylbicycloheptane, diaminooxybicycloheptane, diaminomethyloxybicycloheptane, isophoronediamine, diaminotricyclodecane, diaminomethyltricyclodecane, bis(aminocyclohexyl) methane, bis(aminocyclohexyl) isopropylidene, 6,6′-bis(3-aminophenoxy)-3,3,3′,3′-tetramethyl-1,1′-spirobiindane, and 6,6′-bis(4-aminophenoxy)-3,3,3′,3′-tetramethyl-1,1′-spirobiindane; fluorine-based diamines containing a fluorine atom, such as 2,2′-bis(trifluoromethyl)-4,4′-diaminobiphenyl, 2,2′-bis(3-amino-4-hydroxyphenyl) hexafluoropropane, 2,3,5,6-tetrafluoro-1,4-diaminobenzene, 2,4,5,6-tetrafluoro-1,3-diaminobenzene, 2,3,5,6-tetrafluoro-1,4-benzene(dimethaneamine), 2,2′-difluoro-(1,1′-biphenyl)-4,4′-diamine, 2,2′,6,6′-tetrafluoro-(1,1′-biphenyl)-4,4′-diamine, 4,4′-diaminooctafluorobiphenyl, 2,2-bis(4-aminophenyl) hexafluoropropane, 4,4′-oxybis(2,3,5,6-tetrafluoroaniline), 3,3′-bis(trifluoromethyl)-4,4′-diaminobiphenyl, 4,4′-diamino-2,2′-bis(trifluoromethyl)diphenyl ether, 1,4-bis[4-amino-2-(trifluoromethyl) phenoxy]benzene, 2,2-bis[4-[4-amino-2-(trifluoromethyl) phenoxy]hexafluoropropane, 3,5-diaminobenzene trifluoride, and 4,4-diamino-2-(trifluoromethyl)diphenyl ether; ester bond-containing diamines such as 4-aminophenyl 4-aminobenzoate and bis(4-aminophenyl) terephthalate; and the like. These may be used alone or as a mixture thereof.

As the diamine compounds as the diamine component, 4,4′-diaminodiphenyl ether, 2,2′-dimethylbenzidine, 4,4′-diaminodiphenylmethane, 4,4′-diamino-1,2-diphenylethane, p-phenylenediamine (PPD), and 2,2-bis[4-(4-aminophenoxy)phenyl]propane are preferred, and p-phenylenediamine (PPD) is more preferred.

In the polyimide precursor according to the first embodiment, a group containing an acidic group is contained as at least part of the group represented by X¹ in General Formula (1) above, the group represented by Y¹ in General Formula (1) above, and the terminal group, and the acidic group content ratio is 15×10⁻³ or more. The acidic group content ratio is preferably 16×10⁻³ or more, more preferably 18×10⁻³ or more, still more preferably 20×10⁻³ or more. When the acidic group is contained in such a content ratio, a polyimide film formed from the polyimide precursor according to the first embodiment can contain a predetermined amount of the acidic group, which can increase the charge half-life and reduce the charge decay ratio after 120 seconds.

The acidic group content ratio can be determined from the following equation:

acidic group content ratio={(molar amount of acidic group-containing monomer used to form polyimide precursor)×(the number of acidic groups per molecule of acidic group-containing monomer)}=(molar amount of total monomers forming polyimide precursor)

Here, in the case where a plurality of acidic group-containing monomers each having a different number of acidic groups per molecule is used, the acidic group content ratio of each acidic group-containing monomer may be calculated from the above equation according to the proportion of these acidic group-containing monomers used, and may be used. Moreover, for the tetracarboxylic acid, it is assumed that among the acidic groups in one molecule, four carboxylic acids are reacted with a diamine during polymerization, and are no longer left as the acidic groups when polyimide is prepared. Thus, the number of acidic groups in calculation of the acidic group amount is calculated using a numeric value obtained by subtracting 4 from the number of acidic groups (including acid anhydride groups) in one molecule of the tetracarboxylic acid. In other words, the acidic group content ratio is calculated based on the number of free carboxyl groups which do not contribute to formation of imide bonds. For example, 3, 3′,4,4′-biphenyltetracarboxylic dianhydride has only four carboxyl groups, all of which contribute to formation of imide bonds. Thus, the number of acidic groups is zero in calculation of the acidic group content ratio. While mellitic acid can be in a mixed state of those without an anhydride group, those having one anhydride group, and those having two anhydride groups, typically, four of six carboxyl groups constituting mellitic acid are used to form imide bonds, and two free carboxyl groups are left after the polyimide precursor or the polyimide film is formed. Thus, for mellitic acid, the acidic group content ratio can be calculated where the number of carboxyl groups as the acidic group is considered as two.

Examples of the acidic group include, but not particularly limited to, a carboxyl group (—COOH), a sulfonic acid group (—SO₃H), a phosphonic acid group (—PO₃H₂), and the like. Among these, a carboxyl group and a sulfonic acid group are preferred because they have a high effect of suppressing charge up. Here, the acidic group may be one hydrolyzed to form an acidic group not esterified or the like.

The group represented by X¹ in General Formula (1) above containing a group containing an acidic group can be formed by any method, and examples thereof include a method using a specific tetracarboxylic acid compound as at least part of the tetracarboxylic acid component. Examples of such a specific tetracarboxylic acid compound include compounds having an acidic group such as a carboxyl group other than the carboxyl groups constituting —COOR¹, —COOR², and two amido bonds (—CONH—) in General Formula (1) above (hereinafter, referred to as tetracarboxylic acid compounds having an acidic group). In other words, the tetracarboxylic acid compound having an acidic group is a compound having, in addition to the tetracarboxylic acid structure which contributes to the imidization reaction, an acidic group which does not contribute to an imidization reaction.

Specific examples of the tetracarboxylic acid compound having an acidic group include compounds having a carboxyl group as the acidic group, such as mellitic acid, mellitic anhydride, mellitic acid methyl ester, mellitic acid dimethyl ester, mellitic acid trimethyl ester, mellitic acid ethyl ester, mellitic acid diethyl ester, mellitic acid triethyl ester, mellitic acid propyl ester, mellitic acid dipropyl ester, mellitic acid tripropyl ester, mellitic acid butyl ester, mellitic acid dibutyl ester, mellitic acid tributyl ester, mellitic acid phenyl ester, mellitic acid diphenyl ester, mellitic acid triphenyl ester, and the like. These may be used alone or as a mixture thereof. Among these, mellitic acid and mellitic anhydride are preferred.

Although the amount of the tetracarboxylic acid compound having an acidic group used can be appropriately selected according to the amount of the acidic group contained in the polyimide precursor according to the first embodiment, the amount is preferably 1 mol % or more, more preferably 2 mol % or more, preferably 70 mol % or less, more preferably 60 mol % or less, still more preferably 50 mol % or less, further still more preferably 10 mol % or less, particularly preferably 6 mol % or less in 100 mol % of the total amount of the tetracarboxylic acid component.

The group represented by Y in General Formula (1) above containing a group containing an acidic group can be formed by any method, and examples thereof include a method using an acidic group-containing diamine compound as at least part of the diamine component. Such an acidic group-containing diamine compound can be a compound having an acidic group such as a carboxyl group in addition to the diamine structure, and examples of such compounds having a carboxyl group as the acidic group include 3,5-diaminobenzoic acid (3,5-DABA), 5,5′-methylenebis(2-aminobenzoic acid), 3,3′-diamino-4,4′-dicarboxybiphenyl, 4,4′-diamino-3,3′-dicarboxydiphenylmethane, 3,3′-diamino-4,4′-dicarboxydiphenylmethane, 2,2-bis[4-(4-amino-3-carboxyphenyl)phenyl]propane, and the like. Examples of compounds having a sulfonic acid group as the acidic group include 1,4-phenylenediamine-2-sulfonic acid, 1,3-phenylenediamine-2-sulfonic acid, 3,5-diamino-2, 4,6-trimethylbenzenesulfonic acid, and 4,4′-diaminostilbene-2,2′-disulfonic acid, 4,4′-bis(4-aminophenoxy) biphenyl-3,3′-disulfonic acid, and the like. These may be used alone or as a mixture thereof. Among these, the compound having a carboxyl group as the acidic group is preferably 3,5-diaminobenzoic acid (3,5-DABA), and the compounds having a sulfonic acid group as the acidic group are preferably 1,4-phenylenediamine-2-sulfonic acid and 1,3-phenylenediamine-2-sulfonic acid.

Although the amount of the acidic group-containing diamine compound used can be appropriately selected according to the amount of the acidic group contained in the polyimide precursor according to the first embodiment, the amount is preferably 2 mol % or more, more preferably 3 mol % or more, still more preferably 4 mol % or more, preferably 70 mol % or less, more preferably 60 mol % or less, still more preferably 50 mol % or less, further still more preferably 10 mol % or less, particularly preferably 6 mol % or less in 100 mol % of the total amount of the diamine component.

The terminal group containing a group containing an acidic group can be formed by any method, and examples thereof include a method of relacing part of the tetracarboxylic acid component by a dicarboxylic acid compound having an acidic group or a dicarboxylic anhydride compound having an acidic group. The dicarboxylic acid compound having an acidic group is a compound having, in addition to the dicarboxylic acid structure which contributes to the imidization reaction, an acidic group which does not contribute to an imidization reaction other than the carboxyl groups constituting the dicarboxylic acid structure. The dicarboxylic anhydride compound having an acidic group is a compound having, in addition to the dicarboxylic anhydride structure which contributes to the imidization reaction, an acidic group which does not contribute to an imidization reaction other than the carboxyl groups constituting the dicarboxylic anhydride structure.

Examples of the dicarboxylic acid compound having an acidic group or the dicarboxylic anhydride compound having an acidic group include compounds having a carboxyl group as the acidic group, such as trimellitic acid, trimellitic anhydride, hemimellitic acid, hemimellitic anhydride, and the like. Examples of compounds having a sulfonic acid group as the acidic group include 3-sulfophthalic acid, 4-sulfophthalic acid, 3-sulfophthalic anhydride, 4-sulfophthalic anhydride, and the like. These may be used alone or as a mixture thereof. Among these, compounds having a carboxyl group as the acidic group are preferred, and trimellitic anhydride is preferred.

Although the amount of the dicarboxylic acid compound having an acidic group or the dicarboxylic anhydride compound having an acidic group used can be appropriately selected according to the amount of the acidic group contained in the polyimide precursor according to the first embodiment, the amount thereof in terms of the tetracarboxylic acid is preferably 1 mol % or more, more preferably 1.5 mol % or more, still more preferably 2 mol % or more, preferably 35 mol % or less, more preferably 30 mol % or less, still more preferably 25 mol % or less, particularly preferably 5 mol % or less in 100 mol % of the total of the tetracarboxylic acid component, the dicarboxylic acid compound having an acidic group, and the dicarboxylic anhydride compound having an acidic group, and the amount thereof in terms of the compound is preferably 2 mol % or more, more preferably 3 mol % or more, still more preferably 4 mol % or more, preferably 70 mol % or less, more preferably 60 mol % or less, still more preferably 50 mol % or less, further still more preferably 10 mol % or less, particularly preferably 6 mol % or less in 100 mol % of the total of the tetracarboxylic acid component, the dicarboxylic acid compound having an acidic group, and the dicarboxylic anhydride compound having an acidic group. Note that the dicarboxylic acid compound having an acidic group and the dicarboxylic anhydride compound having an acidic group have a dicarboxylic acid structure but not the tetracarboxylic acid structure as the structure which contributes to the imidization reaction. Thus, the amount added in terms of tetracarboxylic acid is usually a half of the amount added in terms of the compound.

The polyimide precursor according to the first embodiment can be simply prepared by a traditionally known method, for example. Although the method of preparing the polyimide precursor according to the first embodiment is not particularly limited, for example, by reacting substantially equimolar amounts of the tetracarboxylic acid component and the diamine component in a solvent at a relatively low temperature of 100° C. or lower, preferably 80° C. or lower to avoid progression or excessive progression of the imidization reaction, the polyimide precursor can be prepared in the state where it is dissolved in the solvent, that is, in the state of a polyimide precursor solution. The polyimide precursor solution thus prepared may contain, in addition to a polyamic acid as a polyimide precursor, a polyimide precursor or polyimide formed as a result of progression of the imidization reaction and partially or completely imidized.

The polymerization temperature for preparing the polyimide precursor according to the first embodiment is preferably 25° C. or higher and 100° C. or lower, more preferably 40° C. or higher and 80° C. or lower, still more preferably 50° C. or higher and 80° C. or lower. The polymerization time is preferably 0.1 hours or more and 24 hours or less, more preferably 2 hours or more and 12 hours or less. By controlling the polymerization temperature and the polymerization time within the above ranges, a high molecular weight polyimide precursor can be easily prepared with high production efficiency. Although polymerization can also be performed under an air atmosphere, polymerization is suitably performed usually under an inert gas atmosphere, preferably a nitrogen gas atmosphere. The expression “substantially equimolar amounts of the tetracarboxylic acid component and the diamine component” specifically indicates that the molar ratio of these [the total tetracarboxylic acid components/the total diamine components] is 0.90 or more and 1.10 or less, preferably 0.95 or more and 1.05 or less, still more preferably more than 0.98 and 1.04 or less, further still more preferably more than 0.98 and 1.03 or less. In this specification, “substantially equimolar” indicates a molar ratio in the range of more than 0.99 to 1.01, and “equimolar” indicates a molar ratio of 1.00 (significant figure).

When the tetracarboxylic acid component is reacted with the diamine component, usually, a method of adding the diamine component to a polymerization apparatus containing a solvent, and adding the tetracarboxylic acid component after confirming that the diamine component is dissolved can be suitably used.

The solvent used in the reaction of the tetracarboxylic acid component with the diamine component can be any solvent that enables polymerization of the polyimide precursor and can dissolve the polyimide precursor, and may be either a water solvent or an organic solvent. The solvent may be a mixture of two or more solvents, and a mixed solvent of two or more organic solvents or a mixed solvent of water and one or more organic solvents can also be used. Examples of organic solvents include, but not particularly limited to, N, N-dimethylformamide, N, N-dimethylacetamide, N, N-diethylacetamide, N-methyl-2-pyrrolidone, N-ethyl-2-pyrrolidone, 1,3-dimethyl-2-imidazolidinone, N-methylcaprolactam, hexamethylphosphorotriamide, 1,2-dimethoxyethane, bis(2-methoxyethyl) ether, 1,2-bis(2-methoxyethoxy) ethane, tetrahydrofuran, bis[2-(2-methoxyethoxy)ethyl]ether, 1,4-dioxane, dimethyl sulfoxide, dimethyl sulfone, diphenyl ether, sulfolane, diphenylsulfone, tetramethylurea, anisole, m-cresol, phenol, γ-butyrolactone, and the like. In the first embodiment, the solvent used in polymerization of the polyimide precursor can be used as it is as the solvent for the polyimide precursor solution when the polyimide film for a display substrate is produced.

Although not particularly limited, the solids concentration of the polyimide precursor in the polyimide precursor solution is preferably 5% by mass or more and 45% by mass or less, more preferably 58 by mass or more and 40% by mass or less, still preferably more than 10% by mass and 30% by mass or less relative to the total amount of the polyimide precursor and the solvent. When the solids concentration is less than 5% by mass, it may take some time and effort to increase the thickness of the film in some cases. When the solids concentration is more than 45% by mass, the solution viscosity may be excessively increased, leading to a necessity for a special film production apparatus in some cases.

The solution viscosity at 30° C. of the polyimide precursor solution used in the present invention is not limited, but is preferably 1000 Pa·sec or less, more preferably 0.5 Pa·sec or more and 500 Pa·sec or less, still more preferably 1 Pa·sec or more and 300 Pa·sec or less, particularly preferably 2 Pa·sec or more and 200 Pa·sec or less because of suitable handling.

The polyimide precursor solution may contain known additives, such as an amine compound, an additive for promoting the imidization reaction, such as a dehydrating agent, an organic phosphorus-containing compound, the above-mentioned filler, a surfactant, a silane coupling agent, and a leveling agent, as needed.

Examples of the amine compound include substituted or non-substituted nitrogen-containing heterocyclic compounds, N-oxide compounds of the nitrogen-containing heterocyclic compounds, substituted or non-substituted amino acid compounds, aromatic hydrocarbon compounds or aromatic heterocyclic compounds having a hydroxyl group, and the like. Specific examples of the imidization catalyst include imidazole derivatives such as 1,2-dimethylimidazole, N-methylimidazole, 2-methylimidazole, 2-ethyl-4-methylimidazole, 5-methylbenzimidazole, and N-benzyl-2-methylimidazole; substituted pyridine derivatives such as isoquinoline, 3,5-dimethylpyridine, 3,4-dimethylpyridine, 2,5-dimethylpyridine, 2,4-dimethylpyridine, and 4-n-propylpyridine; and the like. Preferably, the amount of the imidization catalyst to be used is 0.01-fold equivalents or more and 2-fold equivalents or less, particularly 0.02-fold equivalents or more and 1-fold equivalents or less relative to amide acid units of the polyamide precursor. Use of the imidization catalyst may improve physical properties of a polyimide film to be obtained, particularly elongation or end tear resistance in some cases.

Examples of other amine compounds include aliphatic tertiary amines such as trimethylamine and triethylenediamine; aromatic tertiary amines such as dimethylaniline; and heterocyclic tertiary amines such as isoquinoline, pyridine, α-picoline, and β-picoline; and the like, and these can be added as needed.

Examples of the dehydrating agent include aliphatic carboxylic anhydrides such as acetic anhydride, propionic anhydride, and butyric anhydride; aromatic carboxylic anhydrides such as benzoic anhydride; and the like.

Examples of the organic phosphorus-containing compound include phosphoric acid esters such as monocaproyl phosphoric acid ester, monooctyl phosphoric acid ester, monolauryl phosphoric acid ester, monomyristyl phosphoric acid ester, monocetyl phosphoric acid ester, monostearyl phosphoric acid ester, monophosphoric acid ester of triethylene glycol monotridecyl ether, monophosphoric acid ester of tetraethylene glycol monolauryl ether, monophosphoric acid ester of diethylene glycol monostearyl ether, dicaproyl phosphoric acid ester, dioctyl phosphoric acid ester, dicapryl phosphoric acid ester, dilauryl phosphoric acid ester, dimyristyl phosphoric acid ester, dicetyl phosphoric acid ester, distearyl phosphoric acid ester, diphosphoric acid ester of tetraethylene glycol mononeopentyl ether, diphosphoric acid ester of triethylene glycol monotridecyl ether, diphosphoric acid ester of tetraethylene glycol monolauryl ether, and diphosphoric acid ester of diethylene glycol monostearyl ether; and amine salts of these phosphoric acid esters. Examples of the amines include ammonia, monomethylamine, monoethylamine, monopropylamine, monobutylamine, dimethylamine, diethylamine, dipropylamine, dibutylamine, trimethylamine, triethylamine, tripropylamine, tributylamine, monoethanolamine, diethanolamine, triethanolamine, and the like.

### <Polyimide Precursor for Display Substrate According to Second Embodiment>

The polyimide precursor for a display substrate according to a second embodiment (hereinafter, referred to as “polyimide precursor according to the second embodiment”) is a polyimide precursor having a structural unit represented by General Formula (2) below, wherein a group represented by General Formula (3) below is contained as Y² in General Formula (2) below:

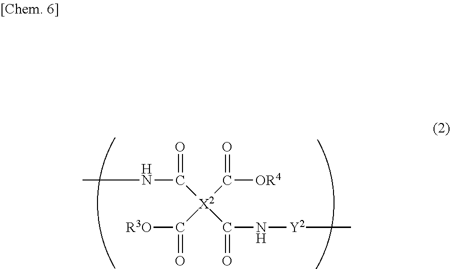

where in General Formula (2), X² is a tetravalent aromatic or aliphatic group, Y² is a divalent aromatic group, and R³ and R⁴ are each independently a hydrogen atom, a C₁ to C₆ alkyl group, or a C₃ to C₉ alkylsilyl group;

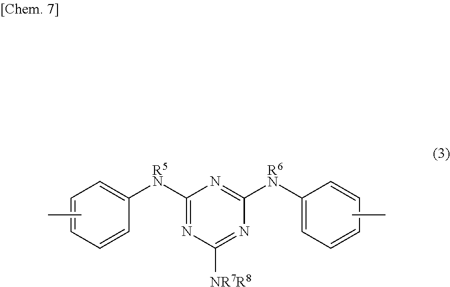

where in General Formula (3), R⁵, R⁶, R⁷, and R⁸ are each independently a hydrogen atom, an optionally substituted C₁ to C₁₂ alkyl group, or an optionally substituted C₆ to C₁₂ aryl group.

In General Formula (2), X² is a residue of a tetracarboxylic acid from which four COOH groups are removed (i.e., a residue of a tetracarboxylic dianhydride from which two carboxylic anhydride groups (CO)₂O are removed), and Y² is a residue of a diamine from which two NH₂ groups are removed. R³ and R⁴ are preferably a hydrogen atom or a C₃ to C₉ alkylsilyl group, more preferably a hydrogen atom.

Examples of the structural unit represented by General Formula (2) include those obtained by reacting a tetracarboxylic acid component with a diamine component to form amido bonds (—CONH—). At this time, by using a triazine structure-containing diamine compound represented by General Formula (4) below as at least part of the diamine component, the polyimide precursor can have the group represented by General Formula (3) as Y² in General Formula (2) above.

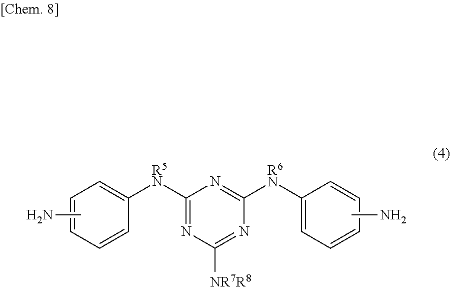

In General Formula (4), R⁵, R⁶, R⁷, and R⁸ are as defined in General Formula (3) above.

The polyimide precursor according to the second embodiment contains the group represented by General Formula (3) as Y² in General Formula (2) above, which can enhance the relative permittivity of a polyimide to be formed from the polyimide precursor according to the second embodiment. The enhanced relative permittivity results in an increase in charge half-life. In particular, the present inventors, who have conducted research, have found that there is a certain correlation between the relative permittivity and the charge half-life, and an enhancement in relative permittivity can increase the charge half-life. Based on such findings, the second embodiment can provide an increased charge half-life and a reduced charge decay ratio after 120 seconds.

In General Formulae (3) and (4), R⁵, R⁶, and R⁷ are each independently a hydrogen atom, an optionally substituted C₁ to C₁₂ alkyl group, or an optionally substituted C₆ to C₁₂ aryl group, preferably a hydrogen atom, a non-substituted C₁ to C₁₂ alkyl group, or a non-substituted C₆ to C₁₂ aryl group, more preferably a hydrogen atom or a non-substituted C₁ to C₄ alkyl group, still more preferably a hydrogen atom.

In General Formulae (3) and (4), R⁸ is a hydrogen atom, an optionally substituted C₁ to C₁₂ alkyl group, or an optionally substituted C₆ to C₁₂ aryl group, preferably a hydrogen atom, a non-substituted C₁ to C₁₂ alkyl group, or a non-substituted C₆ to C₁₂ aryl group, more preferably a hydrogen atom or a non-substituted C₆ to C₁₂ aryl group, still more preferably a phenyl group.

Specific examples of the triazine structure-containing diamine compound represented by General Formula (4) above include 2,4-bis(3-aminoanilino)-6-anilino-1, 3,5-triazine (p-ATDA), 2,4-bis(3-aminoanilino)-6-benzylamino-1, 3,5-triazine, 2,4-bis(3-aminoanilino)-6-naphthylamino-1, 3,5-triazine, 2,4-bis(3-aminoanilino)-6-biphenylamino-1, 3,5-triazine, 2,4-bis(3-aminoanilino)-6-diphenylamino-1,3,5-triazine, 2,4-bis(3-aminoanilino)-6-dibenzylamino-1, 3,5-triazine, 2,4-bis(3-aminoanilino)-6-dinaphthylamino-1, 3,5-triazine, 2,4-bis(3-aminoanilino)-6-N-methylanilino-1, 3,5-triazine, 2,4-bis(3-aminoanilino)-6-N-methylnaphthylamino-1, 3,5-triazine, 2,4-bis(3-aminoanilino)-6-methylamino-1,3,5-triazine, 2,4-bis(3-aminoanilino)-6-ethylamino-1, 3,5-triazine, 2,4-bis(3-aminoanilino)-6-dimethylamino-1, 3,5-triazine, 2,4-bis(3-aminoanilino)-6-diethylamino-1, 3,5-triazine, 2,4-bis(3-aminoanilino)-6-dibutylamino-1, 3,5-triazine, 2,4-bis(3-aminoanilino)-6-amino-1,3,5-triazine, and the like. These may be used alone or as a mixture thereof. Among these, 2,4-bis(3-aminoanilino)-6-anilino-1,3,5-triazine (p-ATDA) is preferred. Use of 2,4-bis(3-aminoanilino)-6-anilino-1, 3,5-triazine (p-ATDA) enables introduction of the group represented by Formula (5) below as Y² in General Formula (2) above.

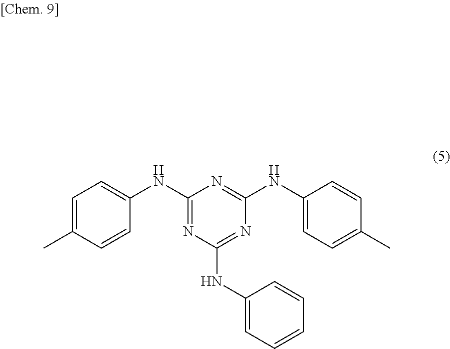

The triazine structure-containing diamine compound represented by General Formula (4) above is used in an amount of preferably 50 to 100 mol %, more preferably 70 to 100 mol %, still more preferably 90 to 100 mol % in 100 mol % of the total amount of the diamine component. In other words, the proportion of the structural unit containing the group represented by General Formula (3) in 100 mol % of the group represented by Y² is preferably 50 to 100 mol %, more preferably 70 to 100 mol %, still more preferably 90 to 100 mol %.

In the second embodiment, a diamine compound other than the triazine structure-containing diamine compound represented by General Formula (4) above may also be used as the diamine component. Examples of such usable diamine compounds include those listed in the first embodiment described above. The acidic group-containing diamine compounds listed in the first embodiment described above may also be used as the diamine compound. In this case, the amount thereof used may be the same as that in the first embodiment described above.

Examples of the tetracarboxylic acid component include aromatic tetracarboxylic dianhydrides, aliphatic tetracarboxylic dianhydrides, and the like. Those listed in the first embodiment described above can be used. As the tetracarboxylic acid component, the tetracarboxylic acid compound having an acidic group, the dicarboxylic acid compound having an acidic group, or the dicarboxylic anhydride compound having an acidic group listed in the first embodiment described above may also be used. In this case, the amount thereof used may be the same as that in the first embodiment described above. The solvent used in the reaction of the tetracarboxylic acid component with the diamine component can also be those listed in the first embodiment described above. In the second embodiment, the solvent used in polymerization of the polyimide precursor can also be used as it is as the solvent for the polyimide precursor solution when the polyimide film for a display substrate is produced.

The polyimide precursor according to the second embodiment can be simply prepared by a traditionally known method, for example. Although the method of preparing the polyimide precursor according to the second embodiment is not particularly limited, the polyimide precursor can be prepared by the same method as that in the first embodiment described above. In the second embodiment, the polyimide precursor can be also prepared in the state where it is dissolved in the solvent, that is, in the state of a polyimide precursor solution. The polyimide precursor solution thus prepared may contain, in addition to a polyamic acid as a polyimide precursor, a polyimide precursor or polyimide formed as a result of progression of the imidization reaction and partially or completely imidized.

In the second embodiment, the solids concentration of the polyimide precursor in the polyimide precursor solution and the solution viscosity to 30° C. of the polyimide precursor solution can also be selected as in the first embodiment, and the polyimide precursor solution may contain known additives, such as an amine compound, an additive for promoting the imidization reaction, such as a dehydrating agent, an organic phosphorus-containing compound, the filler, a surfactant, a silane coupling agent, and a leveling agent, which are listed in the first embodiment, as needed.

### <Polyimide Precursor for Display Substrate According to Third Embodiment>

The polyimide precursor for a display substrate according to the third embodiment is a polyimide precursor-containing composition (hereinafter, referred to as “polyimide precursor composition according to the third embodiment”) containing a polyimide precursor having a structural unit represented by General Formula (6) below and at least one inorganic oxide selected from the group consisting of SiO₂, Al₂O₃, TiO₂, PbTiO₃, CaTiO₃, SrTiO₃, CaZrO₃, and BaTiO₃:

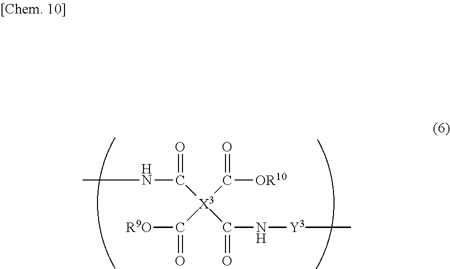

where in General Formula (6), X³ is a tetravalent aromatic or aliphatic group, Y³ is a divalent aromatic group, and R⁹ and R¹⁰ are each independently a hydrogen atom, a C₁ to C₆ alkyl group, or a C₃ to C₉ alkylsilyl group.

In General Formula (6), X³ is a residue of a tetracarboxylic acid from which four COOH groups are removed (i.e., a residue of a tetracarboxylic dianhydride from which two carboxylic anhydride groups (CO)₂O are removed), and Y³ is a residue of a diamine from which two NH₂ groups are removed. R⁹ and R¹⁰ are preferably a hydrogen atom, a C₃ to C₉ alkylsilyl group, more preferably a hydrogen atom.

Examples of the structural unit represented by General Formula (6) which forms the polyimide precursor constituting the polyimide precursor composition according to the third embodiment include those obtained by reacting a tetracarboxylic acid component with a diamine component to form amido bonds (—CONH—).

Usable tetracarboxylic acid compounds constituting the tetracarboxylic acid component and usable diamine compounds constituting the diamine component are the same as those listed in the first embodiment described above. As the tetracarboxylic acid compound, the tetracarboxylic acid compound having an acidic group, the dicarboxylic acid compound having an acidic group, or the dicarboxylic anhydride compound having an acidic group listed in the first embodiment described above may also be used. In this case, the amount thereof used may be the same as those in the first embodiment described above. Further, as the diamine compound, the acidic group-containing diamine compound listed in the first embodiment described above or the triazine structure-containing diamine compound represented by General Formula (4) above listed in the second embodiment described above may also be used. In this case, the amount thereof used can be the same as those in the first embodiment and the second embodiment described above. The solvent used in the reaction of the tetracarboxylic acid component with the diamine component may be those listed in the first embodiment described above. In the third embodiment, the solvent used in polymerization of the polyimide precursor can also be used as it is as the solvent for the polyimide precursor solution when the polyimide film for a display substrate is produced.

The polyimide precursor used in the third embodiment can be simply prepared by a traditionally known method, for example. Although the method of preparing the polyimide precursor used in the third embodiment is not particularly limited, the polyimide precursor can be prepared by the same method as that in the first embodiment described above. In the third embodiment, the polyimide precursor can also be prepared in the state where it is dissolved in the solvent, that is, in the state of a polyimide precursor solution. The polyimide precursor solution thus prepared may contain, in addition to a polyamic acid as a polyimide precursor, a polyimide precursor or polyimide formed as a result of progression of the imidization reaction and partially or completely imidized.

The polyimide precursor composition according to the third embodiment comprises a polyimide precursor and at least one inorganic oxide selected from the group consisting of SiO₂, Al₂O₃, TiO₂, PbTiO₃, CaTiO₃, SrTiO₃, CaZrO₃, and BaTiO₃. In the third embodiment, by compounding such an inorganic oxide, a polyimide film to be formed from the polyimide precursor composition according to the third embodiment can have enhanced relative permittivity. The enhanced relative permittivity results in an increased charge half-life. In particular, the present inventors, who have conducted research, have found that there is a certain correlation between the relative permittivity and the charge half-life, and an enhancement in relative permittivity can increase the charge half-life. Based on such findings, the third embodiment can provide an increased charge half-life and a reduced charge decay ratio after 120 seconds.

The inorganic oxide is preferably CaTio₃, SrTiO₃, CaZrO₃, and BaTiO₃, more preferably BaTiO₃ because these can further enhance the effect of improving the relative permittivity, which can further increase the charge half-life.

The amount of the inorganic oxide used is preferably 30 parts by mass or more, more preferably 40 parts by mass or more, still more preferably 45 parts by mass or more, preferably 100 parts by mass or less, more preferably 90 parts by mass or less, still more preferably 80 parts by mass or less relative to 100 parts by mass of the polyimide precursor. By controlling the amount of the inorganic oxide used within the above ranges, the effect of improving the relative permittivity can be further enhanced, which can further increase the charge half-life. In addition, the charge decay ratio after 120 seconds can be reduced. In particular, while favorable physical properties of the film such as transmittance are maintained, the charge half-life can be further increased, and the charge decay ratio after 120 seconds can be reduced.

In the third embodiment, the inorganic oxide can be compounded with the polyimide precursor, for example, by adding and mixing a predetermined amount of the inorganic oxide to and with the polyimide precursor solution.

In the third embodiment, the solids concentration of the polyimide precursor in the polyimide precursor solution and the solution viscosity to 30° C. of the polyimide precursor solution can be the same as those in the first embodiment. The polyimide precursor solution may also contain known additives, such as an amine compound, an additive for promoting the imidization reaction, such as a dehydrating agent, an organic phosphorus-containing compound, the filler, surfactant, a silane coupling agent, and a leveling agent, which are listed in the first embodiment, as needed.

### <Polyimide Film for Display Substrate>

The polyimide film for a display substrate according to the present invention is a product formed from the above-mentioned polyimide precursor for a display substrate or the polyimide precursor composition for a display substrate.

Since the polyimide film for a display substrate according to the present invention is formed from the polyimide precursor for a display substrate, the polyimide film has a charge half-life of 48 seconds or more or a charge decay ratio after 120 seconds of 63% or less in the measurement of the charge half-life in accordance with JIS L 1094A, and thus, can contribute to a suppression in charge up.

For example, the polyimide film for a display substrate according to the present invention can be produced by a known method using the above-mentioned polyimide precursor solution (a solution containing the polyimide precursor for a display substrate according to the present invention, more specifically, a solution containing the polyimide precursor for a display substrate or the polyimide precursor composition for a display substrate according to the first to third embodiments, which is also referred to as “display substrate formation solution”).

For example, the polyimide film for a display substrate according to the present invention can be produced by a method of applying the polyimide precursor solution onto a support, drying the polyimide precursor solution to form a laminate of the support and the polyimide precursor film, and chemically or thermally imidizing the laminate; a method of applying the polyimide precursor solution onto a support, drying the polyimide precursor solution to form a laminate of the support and the polyimide precursor film, chemically or thermally imidizing the laminate, and then peeling the polyimide film from the support; or a method of applying the polyimide precursor solution onto a support, drying the polyimide precursor solution, peeling the polyimide precursor film from the support to obtain a self-supporting film, fixing the self-supporting film, and chemically or thermally imidizing the self-supporting film.

Although the polyimide precursor solution can be applied onto a support by any method as long as it can form a desired coating, for example, a known method such as spin coating, screen printing, bar coating, electrophoretic deposition, casting, or extrusion molding can be suitably used. Considering the subsequent steps, such as drying and heating, to form the polyimide film, the coating can be formed to have a thickness of about 1 μm to 500 μm.

Although the drying conditions are not particularly limited, the drying temperature can be in the temperature range of, for example, 20° C. or higher and 200° C. or lower, preferably room temperature (25° C.) or higher and 180° C. or lower, more preferably 30° C. or higher and 150° C. or lower. The time for drying is, for example, 1 minute or more and 60 minutes or less, preferably 30 minutes or more and 20 minutes or less although it varies depending on the heating temperature. The heating method is not particularly limited, for example, hot air, infrared light, or the like can be used; the heating method may be performed several times, or may be performed while the temperature is being gradually increased. The drying conditions can be selected considering the properties of the polyimide film in vacuum or an atmosphere of an inert gas such as nitrogen, or air, for example.

Although the support to which the polyimide precursor solution is applied can be any support to which the polyimide precursor solution can be applied and which does not affect the subsequent formation of the polyimide precursor film by drying, heating, and the chemical or thermal imidization reaction, a glass, metal, or plastic substrate is preferably used.

In the present invention, the chemical or thermal imidization can be performed by performing a heat treatment. An example of the thermal imidization will be described: Generally, the highest heating temperature in the heat treatment is 300° C. or higher, preferably 350° C. or higher, more preferably 450° C. or higher, still more preferably 470° C. or higher. The upper limit of the heat treatment temperature can be a temperature which does not cause a reduction in properties of the polyimide film, and is preferably 600° C. or lower, more preferably 550° C. or lower, still more preferably 520° C. or lower. Although the heat treatment can also be performed under an air atmosphere, usually, it is suitably performed under an inert gas atmosphere, preferably a nitrogen gas atmosphere. In the chemical imidization, the heat treatment can be performed on a condition milder than that for the thermal imidization although it depends on the type of an additive such as a chemical imidization catalyst. For example, the heat treatment can be performed in the temperature range of usually 100° C. or higher, preferably 120° C. or higher, more preferably 150° C. or higher, still more preferably 200° C. or higher and usually 360° C. or lower, preferably 300° C. or lower, more preferably 250° C. or lower, still more preferably 220° C. or lower.

The heat treatment for the chemical or thermal imidization may be performed stepwise. For example, it is preferred that a first heat treatment be performed at a relatively low temperature of 100° C. to 170° C. for 0.5 to 30 minutes, a second heat treatment be then performed at a temperature of more than 170° C. to 220° C. or less for about 0.5 to 30 minutes, and subsequently, a third heat treatment be performed at a high temperature of more than 220° C. to less than 350° C. for about 0.5 to 30 minutes. Further, a fourth high-temperature heat treatment can be performed at 350° C. or more to the highest heating temperature. It is preferred that the heat treatment be continuously performed. For example, preferably, a heat treatment can be performed from a relatively low temperature of 100° C. to 170° C. to the highest heating temperature. Although the heating rate is not particularly limited, the heating rate is preferably 1° C./min or more and 30° C./min or less, particularly preferably 2° C./min or more and 20° C./min or less. The ranges above are preferred because foaming caused by a rapid increase in temperature can be suppressed.

### <Display Substrate>

The polyimide film for a display substrate according to the present invention is suitably used in display substrates for displays and touch panels.

The display substrate is formed as follows, for example. Specifically, first, an inorganic gas barrier layer as a gas barrier layer against water vapors and oxygen is formed on the surface of the polyimide film for a display substrate according to the present invention by sputtering, deposition, or a gel-sol method. The inorganic gas barrier layer is formed of SiOₓ or the like, for example. In the next step, an electrically conductive layer made of a conductive substance (such as a metal or a metal oxide, a conductive organic product, or a conductive carbon) is formed thereon. Thereby, a display substrate can be obtained. The electrically conductive layer with a predetermined circuit pattern is formed by a method such as photolithography, a variety of printing methods, or an inkjet method. Subsequently, members for configuring the display, such as elements and semiconductors, may be additionally formed.

The display substrate according to the present invention may be produced as follows: an inorganic gas barrier layer is formed on the surface of the polyimide film formed from the polyimide precursor for a display substrate according to the present invention, and an electrically conductive layer with a circuit pattern is formed; and thereafter, the polyimide film including the inorganic gas barrier layer and the electrically conductive layer formed on the surface thereof is peeled off from the support. The peeling method is not particularly limited. For example, the polyimide film can be peeled by any peeling method such as laser peeling to peel the film by irradiation of the film with laser or the like from the support side or mechanical peeling to mechanically peel the film.

The display substrate according to the present invention thus obtained includes a polyimide film for a display substrate formed from the polyimide precursor for a display substrate according to the present invention, an inorganic gas barrier layer formed on the surface of the polyimide film, and an electrically conductive layer with a circuit pattern formed thereon. Since the display substrate according to the present invention includes a polyimide film for a display substrate formed from the polyimide precursor for a display substrate according to the present invention and the polyimide film for a display substrate according to the present invention has a long charge half-life, such a polyimide film is effective in cancelling charge accumulation at the interface with the inorganic gas barrier layer, and thus contributes to a suppression in charge up.

### EXAMPLES

Hereinafter, the present invention will be more specifically described by way of Examples, Comparative Examples, and Reference Examples, but the present invention is not limited to these.

Measurement methods used in examples below will be shown.

### [Acidic Group Content Ratio]

The total acidic group amount contained in the polyimide film was calculated as follows: initially, for each of acid dianhydride, diamine, tetracarboxylic acid, and a terminating agent as raw materials, the amount of the acidic group contained therein was calculated from the following expression. Note that for tetracarboxylic acid, it was assumed that among the acidic groups in one molecule, four carboxylic acids are reacted with a diamine during polymerization, and are no longer left as the acidic groups when the film is formed, and the number of acidic groups in calculation of the acidic group amount was calculated using a numeric value obtained by subtracting 4 from the number of acidic group in one molecule of the tetracarboxylic acid (including acid anhydride groups).

amount (mol) of acidic group contained in raw material=(amount (mol) of substance in raw material)×(the number of acidic groups in one molecule of raw material)

Next, the total molar amount of acid dianhydride, diamine, tetracarboxylic acid, and the terminating agent excluding the catalyst was regarded as the total amount (mol) of the monomer added, and the content ratio of acidic groups (—COOH group, —SOH groups) contained in the polyimide film was calculated from the expression below:

content ratio of acidic groups contained in film=total amount (mol) of acidic groups contained in raw material/total amount (mol) of monomer added

[Charge Half-Life and Charge Decay Ratio after 120 Seconds in Measurement of Charge Half-Life]

A polyimide film was cut into a size of 55 mm in length and width to prepare a sample piece. Using Static Honestmeter available from SHISHIDO ELECTROSTATIC, LTD., this test piece was subjected to measurement of the charge half-life in accordance with JIS L 1094A by a corona charge method under an environment at 23±2° C. and 50% RH where a voltage of −10 kV was applied for 30 s and the maximum measurement time was 120 s, thereby measuring charge half-life and charge decay ratio after 120 seconds.

### [90° Peel Strength]

The 90° peel strength was measured by the following method. A glass plate including a polyimide film formed thereon was cut into a width of 25 mm to prepare a sample piece. In this sample piece, the test piece was partially peeled to make an end for fixation, and was fixed to a peeling test measurement jig of Tensilon RTF-1350. Then, using a tensile tester, 50 mm or more of the test piece was peeled off at a rate of 50 mm/min, and the load during the peeling was measured. The peel-off load was divided by the peeled length (mm) of the test piece, and the obtained value was defined as 90° peel strength.

The following abbreviations stand for the compounds used in Examples below.
S-BPDA: 3,3′,4,4′-biphenyltetracarboxylic dianhydride
PPD: p-phenylenediamine
p-ATDA: 2,4-bis(3-aminoanilino)-6-anilino-1, 3,5-triazine
3,5-DABA: 3,5-diaminobenzoic acid
MPD: m-phenylenediamine
HAB: 3,3′-dihydroxybenzidine
DATP: 4,4″-diamino-p-terphenyl

### Example 1

34.4734 g of NMP (N-methylpyrrolidone), 1.4340 g of PPD, and 0.0842 g of 3,5-DABA were placed into a reactor equipped with a stirrer and a nitrogen inlet pipe, and were stirred under a nitrogen atmosphere at 50° C. for 30 minutes. Thereafter, 3.9820 g of s-BPDA was added to cause a reaction, preparing a polyimide precursor solution (polyamic acid solution). At this time, the molar ratio of s-BPDA:PPD:3,5-DABA was 100:96:4.

In the next step, the polyamic acid solution prepared in Synthetic Example was applied onto an alkali-free glass wafer by spin coating, and the workpiece was heated at 120° C., 150° C., 200° C., at 250° C. each for 10 minutes, and at 450° C. for 5 minutes to remove the solvent and perform imidization. Thus, a polyimide film having a thickness of 10 μm was obtained. The results of evaluations are shown in Table 1.

### Example 2

A polyimide precursor solution was prepared in the same manner as in Example 1 except that the diamine component and the tetracarboxylic acid components were used in a molar ratio of s-BPDA:trimellitic anhydride:PPD of 98:4:100, and then, removal of the solvent, imidization, and the like were performed in the same manner as in Example 1, obtaining a polyimide film. The results of evaluations are shown in Table 1.

### Example 3

A polyimide precursor solution was prepared in the same manner as in Example 1 except that the diamine component and the tetracarboxylic acid components were used in a molar ratio of s-BPDA:mellitic acid:PPD of 98:2:100, and then, removal of the solvent, imidization, and the like were performed in the same manner as in Example 1, obtaining a polyimide film. The results of evaluations are shown in Table 1.

For the mellitic acid used in Example 3, it was assumed that as a result of a reaction similar to that of the tetracarboxylic acid component, the acidic groups finally left in the film were two carboxylic acids after excluding four carboxylic acids of the tetracarboxylic acid component from six carboxylic acids corresponding to the number of acidic groups of mellitic acid, and the number of acidic groups per molecule of the raw material in calculation of the acidic group content ratio was 2.

### Example 4

A polyimide precursor solution was prepared in the same manner as in Example 1 except that the diamine component and the tetracarboxylic acid component were used in a molar ratio of s-BPDA:p-ATDA of 100:100, and then, removal of the solvent, imidization, and the like were performed in the same manner as in Example 1, obtaining a polyimide film. The results of evaluations are shown in Table 1.

### Example 5

A polyimide precursor solution was prepared in the same manner as in Example 1 except that the diamine component and the tetracarboxylic acid component were used in a molar ratio of s-BPDA:PPD of 100:100.

Then, 50 parts by mass of BaTio₃ particles (trade name “PALCERAM (registered trademark) BTC-4FA”, available from Nippon Chemical Industries Co., Ltd.) relative to 100 parts by mass of the polyimide precursor was added to the polyimide precursor solution, and then, removal of the solvent, imidization, and the like were performed in the same manner as in Example 1, obtaining a polyimide film. The results of evaluations are shown in Table 1.

### Example 6

A polyimide precursor solution was prepared in the same manner as in Example 1 except that the diamine components and the tetracarboxylic acid component were used in a molar ratio of s-BPDA:PPD:1, 4-phenylenediamine-2-sulfonic acid of 100:96:4, and then, removal of the solvent, imidization, and the like were performed in the same manner as in Example 1, obtaining a polyimide film. The results of evaluations are shown in Table 1.

### Comparative Example 1

A polyimide precursor solution was prepared in the same manner as in Example 1 except that the diamine component and the tetracarboxylic acid component were used in a ratio of s-BPDA:PPD of 100:100, and then, removal of the solvent, imidization, and the like were performed in the same manner as in Example 1, obtaining a polyimide film. The results of evaluations are shown in Table 1.

### Comparative Example 2

A polyimide precursor solution was prepared in the same manner as in Example 1 except that the diamine components and the tetracarboxylic acid component were used in a ratio of s-BPDA:PPD:MPD of 100:96:4, and then, removal of the solvent, imidization, and the like were performed in the same manner as in Example 1, obtaining a polyimide film. The results of evaluations are shown in Table 1.

### Comparative Example 3

A polyimide precursor solution was prepared in the same manner as in Example 1 except that the diamine components and the tetracarboxylic acid component were used in a ratio of s-BPDA:PPD:HAB of 100:96:4, and then, removal of the solvent, imidization, and the like were performed in the same manner as in Example 1, obtaining a polyimide film. The results of evaluations are shown in Table 1.

### Comparative Example 4

A polyimide precursor solution was prepared in the same manner as in Example 1 except that the diamine component and the tetracarboxylic acid component were used in a ratio of s-BPDA:DATP of 100:100, and then, removal of the solvent, imidization, and the like were performed in the same manner as in Example 1, obtaining a polyimide film. The results of evaluations are shown in Table 1.

TABLE 1

| | | Exam- | Exam- | Exam- | Exam- | Exam- | Exam- | Comp. | Comp. | Comp. | Comp. |
|---|---|---|---|---|---|---|---|---|---|---|---|
| | | ple 1 | ple 2 | ple 3 | ple 4 | ple 5 | ple 6 | Ex. 1 | Ex. 2 | Ex. 3 | Ex. 4 |
| Composition | s-BPDA | 100 | 98 | 98 | 100 | 100 | 100 | 100 | 100 | 100 | 100 |
| of polyimide | Trimellitic anhydride | | 4 | | | | | | | | |
| (molar ratio) | Mellitic acid | | | 2 | | | | | | | |
| | PPD | 96 | 100 | 100 | | 100 | 96 | 100 | 96 | 98 | |
| | p-ATDA | | | | 100 | | | | | | |
| | 3,5-DABA | 4 | | | | | | | | | |
| | 1,4-Phenylenediamine- | | | | | | 4 | | | | |
| | 2-sulfonic acid | | | | | | | | | | |
| | MPD | | | | | | | | 4 | | |
| | HAB | | | | | | | | | 2 | |
| | DATP | | | | | | | | | | 100 |
| Inorganic | BaTiO₃ | | | | | 50mass % | | | | | |
| oxide | | | | | | | | | | | |
| Results of | Acidic group content | 20 COOH | 20 COOH | 20 COOH | 0 | 0 | 20 SO3H | 0 | 0 | 0 | 0 |
| evaluations | ratio (×10−3) | | | | | | | | | | |
| | in polyimide | | | | | | | | | | |
| | Charge half-life (s) | 55.3 | 68.4 | >120 | 53.9 | 55.8 | >120 | 46.7 | 26.8 | 30.3 | 15.6 |
| | in measurement of | | | | | | | | | | |
| | charge half-life | | | | | | | | | | |
| | Charge decay ratio | 61 | 58 | 38 | 61 | 58 | 46 | 64 | 71 | 70 | 77 |
| | after 120 seconds | | | | | | | | | | |
| | (%) in measurement | | | | | | | | | | |
| | of charge half-life | | | | | | | | | | |

Table 1 shows that polyimide films having a charge half-life of 48 seconds or more and a charge decay ratio after 120 seconds of 63% or less were obtained by containing the acidic group in the polyimide precursor in a predetermined proportion (Examples 1 to 3, and 6), by containing the group represented by General Formula (3) in the polyimide precursor (Example 4), or by containing a predetermined inorganic oxide in the polyimide precursor (Example 5), and due to their long charge half-life and low charge decay ratio after 120 seconds, the polyimide films thus obtained can be used as polyimide films for a display substrate, and can contribute to a suppression in charge up. For the 90° peel strength, it was also verified that the peel strength in Example 1 was 25 mN/mm and was higher than that in Comparative Example 1 (13 mN/mm), and in Examples 2 and 4, the adhesion of the polyimide film was very high so that it was difficult to peel the film in order to set the film in the jig for measurement of the peel strength.

### INDUSTRIAL APPLICABILITY

The polyimide precursor for a display substrate according to the present invention is suitably used in display substrates.

What is claimed is:

1. A polyimide precursor for a display substrate, wherein the polyimide precursor is capable of forming a polyimide film for a display substrate having a charge half-life of 48 seconds or more in measurement of the charge half-life in accordance with JIS L 1094A.

2. A polyimide precursor for a display substrate, wherein the polyimide precursor is capable of forming a polyimide film for a display substrate having a charge decay ratio after 120 seconds of 63% or less in measurement of the charge half-life in accordance with JIS L 1094A.

3. The polyimide precursor for a display substrate according to claim 1, wherein the polyimide precursor is capable of forming a polyimide film for a display substrate having a charge decay ratio after 120 seconds of 63% or less in the measurement of the charge half-life in accordance with JIS L 1094A.

4. The polyimide precursor for a display substrate according to claim 1, wherein the polyimide precursor has a structural unit represented by General Formula (2) below, and contains a group represented by General Formula (3) below as Y² in General Formula (2) below: 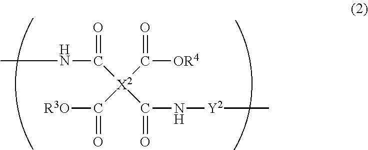
where in General Formula (2), X² is a tetravalent aromatic or aliphatic group, Y² is a divalent aromatic group, and R³ and R⁴ are each independently a hydrogen atom, a C₁ to C₆ alkyl group, or a C₃ to C₉ alkylsilyl group; 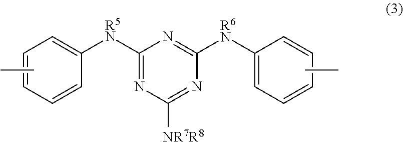
where in General Formula (3), R⁵, R⁶, R⁷, and R⁸ are each independently a hydrogen atom, an optionally substituted C₁ to C₁₂ alkyl group, or an optionally substituted C₆ to C₁₂ aryl group.

5. The polyimide precursor for a display substrate according to claim 4, wherein the polyimide precursor contains a group represented by General Formula (5) below as the group represented by General Formula (3):

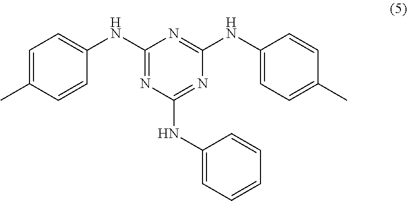

6. The polyimide precursor for a display substrate according to claim 4, wherein the proportion of the structural unit containing the group represented by General Formula (3) as the group represented by Y² in the structural unit represented by General Formula (2) constituting the polyimide precursor for a display substrate is 50 to 100 mol %.

7. A polyimide precursor composition for a display substrate comprising the polyimide precursor for a display substrate according to claim 1, and at least one inorganic oxide selected from the group consisting of SiO₂, Al₂O₃, TiO₂, PbTiO₃, CaTiO₃, SrTiO₃, CaZrO₃, and BaTiO₃,
wherein the polyimide precursor for a display substrate is a polyimide precursor having a structural unit represented by General Formula (6) below: 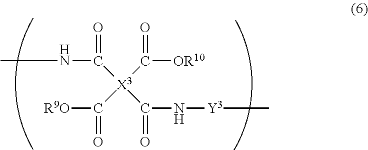
where in General Formula (6), X³ is a tetravalent aromatic or aliphatic group, Y³ is a divalent aromatic group, and R⁹ and R¹⁰ are each independently a hydrogen atom, a C₁ to C₆ alkyl group, or a C₃ to C₉ alkylsilyl group.

8. A polyimide film for a display substrate, which is formed from the polyimide precursor for a display substrate according to claim 1.

9. A display substrate including the polyimide film for a display substrate according to claim 8.

10. A polyimide film for a display substrate, which is formed from the polyimide precursor for a display substrate according to claim 2.

11. A display substrate including the polyimide film for a display substrate according to claim 10.

12. A polyimide film for a display substrate, which is formed from the polyimide precursor composition for a display substrate according to claim 7.

13. A display substrate including the polyimide film for a display substrate according to claim 12.
